(19) United States
(12) Patent Application Publication
(10) Pub. No.: US 2009/0299350 A1
(43) Pub. Date: Dec. 3, 2009

(54) Method for the Medical Treatment of Patients

(76) Inventor: Olaf Schafer, Singen (DE)

Correspondence Address: LAMBERT & ASSOCIATES, P.L.L.C., 92 STATE STREET, BOSTON, MA, 02109-2004, US

(21) Appl. No.: 12/085,240

(22) PCT Filed: Nov. 16, 2006
(86) PCT No.: PCT/EP2006/011003

§ 371 Date: May 26, 2009

(30) Foreign Application Priority Data
Nov. 18, 2005 (DE) 10 2005 055 523.3

(51) Int. Cl. A61B 18/20

(52) U.S. Cl. 606/10

(57) ABSTRACT

Disclosed is a method for the medical treatment of patients by means of a laser beam from a semiconductor laser or diode laser. In said method, the laser beam is to be applied to the area to be treated in the form of pulses.

The invention concerns a method for medical treatment of patients with a laser beam from a semiconductor or diode laser, as well as a corresponding device.

### PRIOR ART

Laser systems now represent an essential tool in medicine. They permit precise, point-like and contactless work. There are a variety of laser systems for medical applications. Of central significance here for each laser is its active medium, which stipulates the emission wavelength and therefore the area of application of the laser in medicine. The choice is essentially made by the wavelength-dependent absorption of the laser radiation in tissue.

Different laser systems are used both in human medicine, as for example, eye surgery, dermatology, plastic surgery, gynecology, neurosurgery, urology and dentistry and in veterinary medicine. In dentistry the laser is used, for example, to treat parodontosis and gum diseases, and as a drill replacement.

The basis of generation of laser radiation is always stimulated emission. By excitation of atoms or molecules in a laser-active medium, occupation of higher energy levels occurs, which are responsible for the laser transition. If excitation is strong enough in order to generate (pump) an overpopulation of the upper laser level, one speaks of population inversion. Ultimately, as a result of spontaneous emission transition to stimulated emission, an artificially generated depopulation of the upper laser level and emission of laser beams occur.

The method with which the laser medium is excited depends on the employed laser medium. The three most essential excitation types are
gas discharge, i.e., plasma formation in gas lasers
optical pumping in solid laser systems
electrical pumping in diode lasers

Semiconductors are used in diode lasers as active medium, which during excitation emit a coherent beam in the visible and near-infrared spectral range. The energy states of the electrons in semiconductors are not sharp, as in free atoms, but produce broad bands. The ground state is formed by the valence band, the excited state by the conduction band. Excitation ordinarily occurs at the so-called pn-transition after application of external voltage. The electrons are passed from the valence band into the conduction band, which leads to population inversion. During subsequent stimulated emission they return to the valence band and then emit light. The emission wavelength depends on the energy distance between the valence and conduction band, the band distance being obtained from selection of appropriate semiconductor compounds.

Diode lasers have been used in medicine since the mid-90s. The main areas of application are coagulation (hemostasis), surgery (ablation of soft tissue) and elimination of disease pathogens. In addition, high-powered diode lasers, as described in DE 10 2004 006 932, can also be used for hair removal (epilation).

Diode lasers have been successfully used in dentistry since the mid-90s for surgical incision, killing of germs, soft laser therapy and bleaching of teeth. Whereas a power of 1 to 3 watts is sufficient for the mentioned applications, the initial power of the device in surgical incisions influences the cutting speed. A higher output power is therefore desirable here. The devices offered on the market, however, only reach an increase in cutting speed by output powers to 15 watts.

Diode lasers are available in several discrete wavelengths. For example, wavelengths of 635 nm (visible, red), 810 nm, 940 nm and 980 nm (all infrared, invisible) are known with sufficient power for the described situations. There are additional wavelengths that have insufficient output power for the described applications.

Diode lasers of 810 nm and 980 nm are mostly used in dentistry.

The action principle of these diode lasers on the patient is absorption of laser light by biological tissue. Absorption by water is insignificant in this wavelength range (0.01 to 0.1%). The essential action mechanism develops by absorption of laser light by melanin (skin) or hemoglobin (red blood dye). Surgical incisions with a diode laser therefore require tissue with good blood supply.

Unlike other laser systems (gas or solid lasers), which are capable of storing optical energy and releasing it in a short pulse, diode lasers can only be switched on and off like an incandescent lamp. Whereas a solid laser, like an Nd:YAG laser, can release an energy pulse of more than one joule in a short time (typically a few μs), which corresponds to a peak power of several thousand watts, the diode laser can only be switched on with a maximum power (typically 2 to 15 watts). If this occurs in a period of few μS, as in Nd:YAG lasers, energies of only a few mJ are reached. Diode lasers are therefore mostly operated in medical applications continuously (CW) or with relatively long quasipulses (a few ms).

For the thermal effect on biological tissue, however, the energy release behavior over time is enormously important. If the laser energy is released in a short pulse, the thermal load on the tissue is lower. With extremely short pulses in the ns-fs range (femtosecond lasers), thermal energy transferred to the tissue no longer occurs at all.

In diode lasers with increasing laser power, with which the cutting speed in surgical applications can be increased, the thermal load leads to burning of the tissue. This is referred to as carbonization and means: carbonized tissue. Because of this wound healing is disturbed and toxic byproducts occur. The thermal effect of carbonization therefore limits the maximum useable laser power and therefore the cutting speed.

Another aspect of previously known diode lasers is poor coupling of the laser radiation into the tissue. The less hemoglobin contained in the tissue, the less laser radiation is absorbed by the tissue. In dental practice this is often a problem because blood supply is reduced by anesthetics. The laser beam then often shows no or only a weak reaction on the irradiated tissue.

### Task

The task of the present invention is to make a diode laser more appropriate for medical application.

### Solution of the Task

Applying the laser beam in the form of pulses on the area being treated leads to solution of the task.

This means the pulse behavior of solid lasers, like the Nd:YAG laser, is stimulated with a diode laser in order to eliminate the effects described above.

One requirement for such a diode laser system is a laser source that has sufficient power. For this purpose at least 5 watts, preferably 25 to 50 watts permanent power should be available. The higher the maximum output power, the higher the pulse energy at a specified time interval. In addition, the laser source must have the correct wavelength. The range between 700 to 1050 nm works here. Laser sources with 810 nm are preferably used because absorption in hemoglobin is very good here. The same also applies for a laser source with a wavelength of 940±10 nm.

The switch-on time of the laser should lie in the range from 2 to 500 μs, preferably 10 to 50 μs and the pause time between two pulses should be greater than the pulse time, preferably 2 to 5 times as long as the pulse time so that surgical cutting with significantly increased speed occurs without interfering carbonization effects. Another observation is that coupling into completely anemic tissue without blood supply even occurs.

The electronic control that furnishes the current for the laser diode is set in a preferred practical example permanently during operation at a value that is just below the value at which the laser diode begins to generate laser energy. The control is briefly induced to switch on the maximum admissible current of the laser diode. This process is periodically repeated.

The best results can be achieved with rectangular pulse forms. Because of restrictions of power supply, rectangular pulse forms, however, are not possible in very short pulses (<50 μs). Because of the finite rise and fall times the pulse will be Gaussian or bell-shaped. An essential point for a Gaussian pulse is a steeply rising flank in order to quickly reach the maximum pulse energy.

Quartz fibers are often used to apply laser radiation. The smaller the diameter of the quartz fiber, the greater effect on the tissue because the power density in thinner fibers increases quadratically.

For application of laser light a hand piece is situated on the distal end of the transmission fiber, which permits the physician to apply the laser energy in targeted fashion. In a special variant this hand piece is designed in two parts and provided with coupling optics. In this way transmission fibers and application fibers are separated. Because of this the entire fiber need not be replaced during damage or soiling, but only the application part.

A useful supplement to the described system is expansion by a second laser source, preferably an Er:YAG laser source. All conceivable applications could be conducted in dentistry with it.

### DESCRIPTION OF THE FIGURES

Additional advantages, features and details of the invention are apparent from the following description of the preferred practical examples and with reference to the drawing; in the drawing

FIG. 1 shows a diagram-like depiction of a possible layout of the method according to the invention;

FIG. 2 shows a diagram-like depiction of another possible variant of the method according to the invention;

FIG. 3 shows another diagram-like depiction of one possible variant of the method according to the invention.

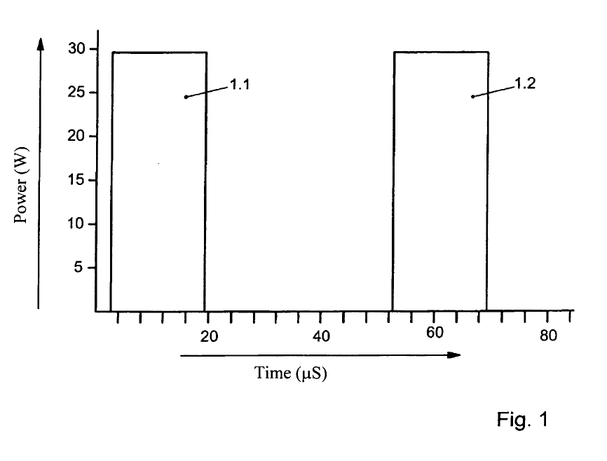

According to FIG. 1 the energy with which a diode laser according to the invention is operated in watts is plotted versus time. It is then recognizable that the laser beam is applied in the form of pulses 1.1 and 1.2. A pulse-free space is situated between pulses 1.1 and 1.2. During this time no application of the laser beam on the area of the patient being treated occurs. Pulses 1.1 and 1.2 are emitted with about 30 watts, the duration of each pulse 1.1 and 1.2 amounts to about 16 μs and the time between pulses 1.1 and 1.2 is about 32 μs. Pulses have a largely rectangular shape.

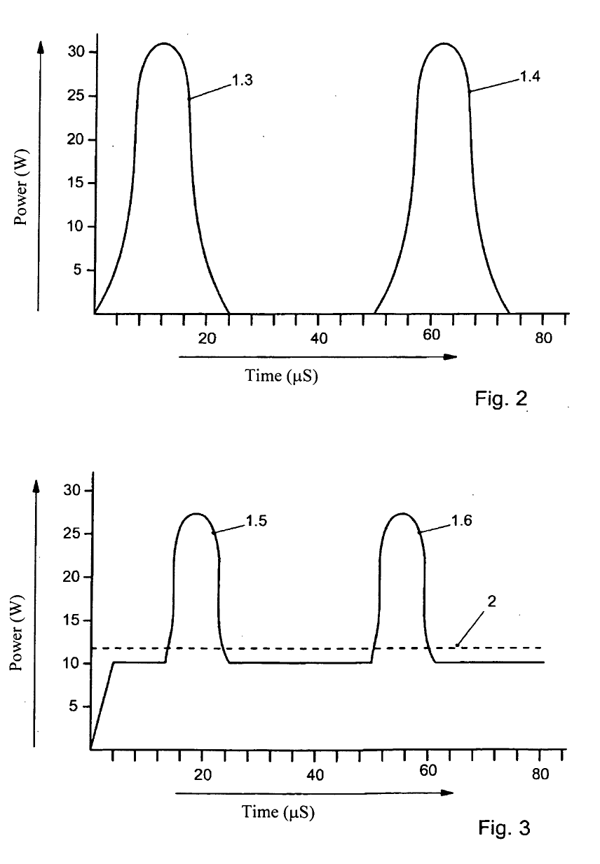

Since the rectangular shape can only be achieved with difficulty in a diode laser, generally the pulses 1.3 and 1.4 according to the method of the invention must be emitted in the form of a Gaussian curve. This is shown in FIG. 2. The rise in each case should occur by more than 0.1 watt per μs so that a relatively steep bell curve is formed. The drop runs similarly.

A particular preferred variant of the method according to the invention is shown in FIG. 3. Here the current and energy are always kept just below a threshold value 2 at which the laser would go into operation. Because of this the time to release of pulses 1.5 and 1.6 is significantly shortened so that a steeper rise of the bell shape of the curve is possible, which closely approaches the rectangular shape.

1-19. (canceled)

20. A method for the medical treatment of an individual using at least one diode laser, wherein the laser includes an active medium, and wherein the laser is powered by electrical current, the steps comprising:
(a) selecting the active medium for use with the laser;
(b) selecting the amount of electrical current for use with the laser;
(c) selecting a wavelength of the laser for treatment of an individual;
(d) selecting a switch-on time of the laser for delivering a pulse from the laser to an individual;
(e) selecting a pause time before delivery of another pulse from the laser to an individual as described in step (d);
(f) delivering a pulse from the laser to an individual for the time specified in step (d);
(g) pausing between the delivery of the next pulse from the laser to an individual for the time specified in step (e); and
(h) repeating steps (f)-(g) until treatment of the individual is complete.

21. The method of claim 20, wherein the active medium is a semiconductor.

22. The method of claim 21, wherein the amount of electrical current utilized with the laser is at least ten watts.

23. The method of claim 22, wherein the amount of electrical current utilized with the laser is between twenty-five and fifty watts.

24. The method of claim 23, wherein the switch-on time of the laser for delivery of a pulse is between two microseconds and five hundred microseconds.

25. The method of claim 24, wherein the switch-on time of the laser for delivery of a pulse is between ten microseconds and fifty microseconds.

26. The method of claim 25, wherein the pause time between deliveries of a pulse from the laser is between two to five times greater than the switch-on time for the pulse.

27. The method of claim 26, wherein the amount of electric current utilized with the laser is below the threshold requirement at which the laser begins to operate.

28. The method of claim 27, wherein the laser further comprises a transmission medium for transmission of the laser.

29. The method of claim 28, wherein the transmission medium is a quartz glass fiber.

30. The method of claim 29, wherein the diameter of the fiber is between one hundred micrometers and four hundred micrometers.

31. The method of claim 30, wherein the laser possesses a wavelength between seven hundred nanometers and one thousand fifty nanometers during delivery of a pulse to an individual.

32. The method of claim 31, wherein the laser includes a front portion and a back portion, wherein the front portion is releaseably attachable to the laser for replacement of the fibers.

33. The method of claim 32, wherein the back portion of the laser further comprises at least two optical elements for coupling of the laser into the fibers.

34. The method of claim 33, wherein a second laser source is assigned to the laser.

35. The method of claim 34, wherein the second laser source is an Er:YAG laser.

36. The method of claim 35, wherein the second laser source possesses a wavelength of two thousand nine hundred and forty nanometers.

37. A method for the medical treatment of an individual, the steps comprising:
(a) applying a laser output from a semiconductor on a first individual pulse, wherein the wave form for said pulse is substantially rectangular.

38. The method of claim 37, wherein the wave form for said pulse is substantially parabolic.

39. The method of claim 37, wherein the wave form for said pulse is substantially semi-parabolic.
